(19) United States
(12) Patent Application Publication
(10) Pub. No.: US 2005/0058628 A1
(43) Pub. Date: Mar. 17, 2005

(54) Nuclear reprogramming of cells for therapeutic use

(76) Inventor: Jan Remmereit, Volda (NO)

Correspondence Address: J. Mitchell Jones;MEDLEN & CARROLL, LLP, Suite 350, 101 Howard Street, San Francisco, CA, 94105, US

(21) Appl. No.: 10/867,544

(22) Filed: Jun. 14, 2004

Related U.S. Application Data
(60) Provisional application No. 60/477,667, filed on Jun. 11, 2003.

(51) Int. Cl. A61K 45/00; C12N 5/08

(52) U.S. Cl. 424/93.7; 435/368

(57) ABSTRACT

The present invention relates to cells that are reprogrammed by exposure to cytoplasm from other cells, and in particular to stem cells that are forced to adopt a particular fate by exposure to differentiated cell cytoplasm.

This application claims the benefit of U.S. provisional application 60/477,667, filed Jun. 11, 2003.

### FIELD OF THE INVENTION

The present invention relates to cells that are reprogrammed by exposure to cytoplasm from other cells, and in particular to cells that are reprogrammed by exposure to stem cell cytoplasm and to stem cells that are forced to adopt a particular fate by exposure to differentiated cell cytoplasm.

### BACKGROUND OF THE INVENTION

Much interest is currently being expressed in the use of stem cells for a variety of tissue replacement therapies. These therapies may be effective for millions of people suffering from diseases ranging from diabetes and sclerosis of the liver to Parkinson's disease and multiple sclerosis.

Stem cells may be derived from two sources, differentiated cells and embryos. For example, U.S. Pat. No. 5,843,780 to Thompson describes the production of stem cell lines from human embryos. However, this approach may be eventually limited due to ethical concerns relating to the use of human embryos. In an attempt to overcome these ethical concerns, PCT publications WO 00/52145 and WO 01/00650 describe the use of cells from adult humans in a nuclear transfer procedure to produce stem cell lines. However, this method met with low efficiency and would theoretically require the production of individualized stem cells lines that could vary greatly in terms of function and totipotency depending on the source of the donor cell.

Examples of adult stem cells include hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

Once stem cells are obtained, they generally must be forced to adopt a particular developmental fate in order to be useful for therapy. Differentiation of stem cells most often requires exposure of the cells to various growth factors and chemicals in an attempt to activate transcription of desired tissue-specific genes. However, the exact cocktail of factors required and the order in which the cells must be exposed to the factors requires laborious experimentation. Methods for encouraging the differentiation of stem cells into particular fates are known in the art and include those described in Palacios et al., Proc. Natl. Acad. Sci. USA 92:7530-37 (1995); Pederson, J. Repro. Fert. Dev. 6:543-55 (1994); Bain et al., Dev. Biol. 168:342-57 (1995); and U.S. Pat. Nos. 5,827,740; 6,149,906; 6,022,540; 6,030,836; 6,060,052; 6,033,906; 6,001,654; and 5,968,501; all of which are incorporated herein by reference in their entirety.

What is needed in the art is a method for causing a stem cell to adopt a particular developmental fate. Such methods could preferably be used to program cells to adopt virtually any developmental fate. Additionally, methods are needed for inducing cells to differentiate so that they may later be reprogrammed to a different developmental fate.

### SUMMARY OF THE INVENTION

The present invention relates to cells that are reprogrammed by exposure to cytoplasm from other cells, and in particular to cells that are reprogrammed by exposure to stem cell cytoplasm and to stem cells that are forced to adopt a particular fate by exposure to differentiated cell cytoplasm.

In some embodiments, the present invention provides compositions comprising isolated cells and a stem cell extract. The present invention is not limited to stem cell extracts prepared from any particular source. Indeed, the stem cell extracts can be prepared from a variety of sources, including, but not limited to, human stem cells and non-human stem cells. The present invention is not limited to cells isolated from any particular source. Indeed, the cells can be isolated from a variety of sources, including, but not limited to cells from a human patient. The present invention is not limited to any particular type of isolated cells. Indeed, the present invention contemplates the use of a variety of cells types, including, but not limited to, neural cells, hematopoietic cells, mesenchymal cells, endodermally derived cells, ectodermally derived cells and mesodermally derived cells. In some embodiments, the composition further comprises telomerase or nucleic acid encoding telomerase.

In other embodiments, the present invention provides compositions comprising isolated stem cells and an extract of a differentiated cell line. In some embodiments, the isolated stem cells are human stem cells. In other embodiments, the isolated stem cells are non-human stem cells. The present invention is not limited to any particular differentiated cell lines. Indeed, the use of extracts from a variety of differentiated cell lines is contemplated, including, but not limited to, neural cell lines, hematopoietic cell lines, mesenchymal cell lines, endodermal cell lines, ectodermal cell lines and mesodermal cell lines. In some embodiments, the composition further comprises telomerase or nucleic acid encoding telomerase.

In still other embodiments, the present invention provides methods comprising: a) providing isolated cells and a stem cell extract; and b) treating the isolated cells with the stem extract under conditions such that the isolated cells are reprogrammed to an earlier differential fate. The present invention is not limited to stem cell extracts prepared from any particular source. Indeed, the stem cell extracts can be prepared from a variety of sources, including, but not limited to, human stem cells and non-human stem cells. The present invention is not limited to cells isolated from any particular source. Indeed, the cells can be isolated from a variety of sources, including, but not limited to cells from a human patient. The present invention is not limited to any particular type of isolated cells. Indeed, the present invention contemplates the use of a variety of cells types, including, but not limited to, neural cells, hematopoietic cells, mesenchymal cells, endodermally derived cells, ectodermally derived cells and mesodermally derived cells. In some embodiments, the treating step further comprising permeabilizing the isolated cells. In other embodiments, the methods comprise the further step of adding telomerase or a nucleic acid encoding telomerase to the stem cell extract.

In still other embodiments, the present invention provides methods comprising: a) providing isolated stem cells and an extract of a differentiated cell line; and b) treating the isolated stem cells with the extract of a differentiated cell line under conditions such that the isolated stem cells adopt a differential fate approximating the differential fate of the differentiated cell line. In some embodiments, the isolated stem cells are human stem cells. In other embodiments, the isolated stem cells are non-human stem cells. The present invention is not limited to any particular differentiated cell lines. Indeed, the use of extracts from a variety of differentiated cell lines is contemplated, including, but not limited to, neural cell lines, hematopoietic cell lines, mesenchymal cell lines, endodermal cell lines, ectodermal cell lines and mesodermal cell lines. In some embodiments, the treating step further comprises permeabilizing the isolated cells. In other embodiments, the methods comprise the further step of adding telomerase or a nucleic acid encoding telomerase to the stem cell extract.

In some embodiments, the present invention provides reprogrammed cells produced by the methods described above. In further embodiments, the present invention provides organs comprising the reprogrammed cells. In some embodiments, the present invention provides differentiated stem cells produced by the methods described above. In further embodiments, the present invention provides organs comprising the differentiated stem cells.

In some embodiments, the present invention provides methods comprising: a) providing isolated cells, a stem cell extract, and a differentiated cell line extract; b) treating the isolated cells with the stem extract under conditions such that the isolated cells are reprogrammed to an earlier differential fate; and c) treating the reprogrammed cells with the differentiated cell line extract under conditions such that the reprogrammed cells adopt a differential fate approximating the differential fate of the differentiated cell line.

In still further embodiments, the present invention provides methods comprising: a) providing isolated cells and a solution comprising telomerase or a nucleic acid encoding telomerase; b) permeabilizing the isolated cells in the solution; and c) culturing the isolated cells, wherein the telomers of the cells are lengthened. In some embodiments, the isolated cells are human cells. In some preferred embodiments, the telomerase is human telomerase. In some embodiments, the cells are genetically modified. The present invention further provides cells produced by the foregoing method and organs comprising the cell.

### DEFINITIONS

As used herein, the term “mesodermal cell line” means a cell line displaying phenotypic characteristics associated with mesodermal cells.

As used herein, the term “endodermal cell line” means a cell line displaying phenotypic characteristics normally associated with endodermal cells.

As used herein, the term “neural cell line” means a cell line displaying characteristics normally associated with neural cell lines. Examples of such characteristics include, but are not limited to, expression of GFAP, neuron-specific enolase, Neu-N, neurofilament-N, or tau.

As used herein, the term “pluripotent” means the ability of a cell to differentiate into the three main germ layers: endoderm, ectoderm, and mesoderm.

As use herein, the term “embryonic human stem cells” means pluripotent cells derived from an embryo.

As used herein, the term “stem cell extract” refers to an extract prepared from stem cells (e.g., embryonic stem cells, neural stem cells, hematopoietic stem cells, or mesenchymal stem cells).

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the term “recombinant DNA molecule” as used herein refers to a DNA molecule that is comprised of segments of DNA joined together by means of molecular biological techniques.

As used herein, the term “purified” or “to purify” refers to the removal of contaminants from a sample.

As used herein, the term “exogenous gene” means a gene that is not normally present in a host cell or organism or is artificially introduced into a host cell or organism.

As used herein, the term “negative selectable marker” refers to a gene that encodes a protein that allows for negative selection. An example of a negative selectable maker is the thymidine kinase gene, which allows for selection with gancyclovir.

As used herein, the term “knock-out mutation” means any mutation that disrupts the function of a product of a wild-type gene. Accordingly, knock-out mutations can be insertional mutations, deletion mutations, point mutations, or frameshift mutations. Knock-out mutations can introduced by a variety of methods including, but not limited to, homologous recombination and transposon insertion.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

### DESCRIPTION OF THE INVENTION

The present invention relates to cells that are reprogrammed by exposure to cytoplasm from other cells, and in particular to cells that are reprogrammed by exposure to stem cell cytoplasm and to stem cells that are forced to adopt a particular fate by exposure to differentiated cell cytoplasm.

A. Sources of Cells and Extracts for Programming or Reprogramming

The present invention contemplates that a variety of cells may be programmed or reprogrammed according to the methods of the present invention, or be used as a source of cytoplasmic extracts for reprogramming target cells. In some embodiments, embryonic stem cells are programmed to adopt particular developmental fates. In other embodiments, adult stem cells are programmed and/or reprogrammed to adopt certain development fates. In still other embodiments, differentiated cells are de-differentiated and/or reprogrammed according to the methods of the present invention.

Primate embryonic stem cells may be preferably obtained by the methods disclosed in U.S. Pat. Nos. 5,843,780 and 6,200,806, each of which is incorporated herein by reference. Primate (including human) stem cells may also be obtained from commercial sources such as WiCell, Madison, Wis. A preferable medium for isolation of embryonic stem cells is “ES medium.” ES medium consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Preferably, fetal bovine serum batches are compared by testing clonal plating efficiency of a low passage mouse ES cell line (ESⱼₜ₃), a cell line developed just for the purpose of this test. FBS batches must be compared because it has been found that batches vary dramatically in their ability to support embryonic cell growth, but any other method of assaying the competence of FBS batches for support of embryonic cells will work as an alternative.

Primate ES cells are isolated on a confluent layer of murine embryonic fibroblast in the presence of ES cell medium. Embryonic fibroblasts are preferably obtained from 12 day old fetuses from outbred CF1 mice (SASCO), but other strains may be used as an alternative. Tissue culture dishes are preferably treated with 0.1% gelatin (type I; Sigma).

For rhesus monkey embryos, adult female rhesus monkeys (greater than four years old) demonstrating normal ovarian cycles are observed daily for evidence of menstrual bleeding (day 1 of cycle=the day of onset of menses). Blood samples are drawn daily during the follicular phase starting from day 8 of the menstrual cycle, and serum concentrations of lutenizing hormone are determined by radioimmunoassay. The female is paired with a male rhesus monkey of proven fertility from day 9 of the menstrual cycle until 48 hours after the lutenizing hormone surge; ovulation is taken as the day following the lutenizing hormone surge. Expanded blastocysts are collected by non-surgical uterine flushing at six days after ovulation. This procedure routinely results in the recovery of an average 0.4 to 0.6 viable embryos per rhesus monkey per month, Seshagiri et al. Am J Primatol 29:81-91, 1993.

For marmoset embryos, adult female marmosets (greater than two years of age) demonstrating regular ovarian cycles are maintained in family groups, with a fertile male and up to five progeny. Ovarian cycles are controlled by intramuscular injection of 0.75 g of the prostaglandin PGF2a analog cloprostenol (Estrumate, Mobay Corp, Shawnee, Kans.) during the middle to late luteal phase. Blood samples are drawn on day 0 (immediately before cloprostenol injection), and on days 3, 7, 9, 11, and 13. Plasma progesterone concentrations are determined by ELISA. The day of ovulation is taken as the day preceding a plasma progesterone concentration of 10 ng/ml or more. At eight days after ovulation, expanded blastocysts are recovered by a non-surgical uterine flush procedure, Thomson et al. “Non-surgical uterine stage preimplantation embryo collection from the common marmoset,” J Med Primatol, 23:333-336 (1994). This procedure results in the average production of 1.0 viable embryos per marmoset per month.

The zona pellucida is removed from blastocysts by brief exposure to pronase (Sigma). For immunosurgery, blastocysts are exposed to a 1:50 dilution of rabbit anti-marmoset spleen cell antiserum (for marmoset blastocysts) or a 1:50 dilution of rabbit anti-rhesus monkey (for rhesus monkey blastocysts) in DMEM for 30 minutes, then washed for 5 minutes three times in DMEM, then exposed to a 1:5 dilution of Guinea pig complement (Gibco) for 3 minutes.

After two further washes in DMEM, lysed trophectoderm cells are removed from the intact inner cell mass (ICM) by gentle pipetting, and the ICM plated on mouse inactivated (3000 rads gamma irradiation) embryonic fibroblasts. After 7-21 days, ICM-derived masses are removed from endoderm outgrowths with a micropipette with direct observation under a stereo microscope, exposed to 0.05% Trypsin-EDTA (Gibco) supplemented with 1% chicken serum for 3-5 minutes and gently dissociated by gentle pipetting through a flame polished micropipette.

Dissociated cells are replated on embryonic feeder layers in fresh ES medium, and observed for colony formation. Colonies demonstrating ES-like morphology are individually selected, and split again as described above. The ES-like morphology is defined as compact colonies having a high nucleus to cytoplasm ratio and prominent nucleoli. Resulting ES cells are then routinely split by brief trypsinization or exposure to Dulbecco's Phosphate Buffered Saline (without calcium or magnesium and with 2 mM EDTA) every 1-2 weeks as the cultures become dense. Early passage cells are also frozen and stored in liquid nitrogen.

The methods of the present invention are not limited to the use of primate embryonic stem cells. Indeed, the use of embryonic stem cells from other species are contemplated, including, but not limited to mice, rats, pigs, cattle and sheep. Methods for obtaining pluripotent cells from these species have been previously described. See, e.g., U.S. Pat. Nos. 5,453,357; 5,523,226; 5,589,376; 5,340,740; and 5,166,065 (all of which are specifically incorporated herein by reference); as well as, Evans, et al., Theriogenology 33(1):125-128, 1990; Evans, et al., Theriogenology 33(1):125-128, 1990; Notarianni, et al., J. Reprod. Fertil. 41(Suppl.):51-56, 1990; Giles, et al., Mol. Reprod. Dev. 36:130-138, 1993; Graves, et al., Mol. Reprod. Dev. 36:424-433, 1993; Sukoyan, et al., Mol. Reprod. Dev. 33:418-431, 1992; Sukoyan, et al., Mol. Reprod. Dev. 36:148-158, 1993; Iannaccone, et al., Dev. Biol. 163:288-292, 1994; Evans & Kaufman, Nature 292:154-156, 1981; Martin, Proc Natl Acad Sci USA 78:7634-7638, 1981; Doetschmanet al. Dev Biol 127:224-227, 1988); Gileset al. Mol Reprod Dev 36:130-138, 1993; Graves & Moreadith, Mol Reprod Dev 36:424-433, 1993 and Bradley, et al., Nature 309:255-256, 1984.

The present invention also contemplates the use of non-embryonic stem cells. Mesenchymal stem cells (MSCs) can be derived from marrow, periosteum, dermis and other tissues of mesodermal origin (See, e.g., U.S. Pat. Nos. 5,591,625 and 5,486,359, each of which is incorporated herein by reference). MSCs are the formative pluripotential blast cells that differentiate into the specific types of connective tissues (i.e. the tissues of the body that support the specialized elements; particularly adipose, areolar, osseous, cartilaginous, elastic, marrow stroma, muscle, and fibrous connective tissues) depending upon various in vivo or in vitro environmental influences. Although these cells are normally present at very low frequencies in bone marrow, various methods have been described for isolating, purifying, and greatly replicating the marrow-derived mesenchymal stems cells in culture, i.e. in vitro (See also U.S. Pat. Nos. 5,197,985 and 5,226,914 and PCT Publication No. WO 92/22584, each of which are incorporated herein by reference).

Various methods have also been described for the isolation of hematopoietic stem cells (See, e.g., U.S. Pat. Nos. 5,061,620; 5,750,397; 5,716,827 all of which are incorporated herein by reference). It is contemplated that the methods of the present invention can be used to produce lymphoid, myeloid and erythroid cells from hematopoietic stem cells. The lymphoid lineage, comprising B-cells and T-cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies in the blood stream, provides protection against neoplastic cells, scavenges foreign materials in the blood stream, produces platelets, and the like. The erythroid lineage provides the red blood cells, which act as oxygen carriers.

The present invention also contemplates the use of neural stem cells, which are generally isolated from developing fetuses. The isolation, culture, and use of neural stem cells are described in U.S. Pat. Nos. 5,654,183; 5,672,499; 5,750,376; 5,849,553; and 5,968,829, all of which are incorporated herein by reference. It is contemplated that the methods of the present invention can use neural stem cells to produce neurons, glia, melanocytes, cartilage and connective tissue of the head and neck, stroma of various secretory glands and cells in the outflow tract of the heart.

In still further embodiments, it is contemplated that the methods of the present invention can be used to reprogram cells derived from a human or animal patient. For example, cells of endodermal origin (e.g., cells from the intestine, liver, pancreas, duodenum, small intenstine, lungs, gall bladder, etc.), ectodermal origin (e.g., cells from the skin, cornea, lens, and cranial ganglia), and mesodermal origin (e.g., cells from the urinary system including pronephros, mesonephros and metanephros cells, skeletogenous tissue and voluntary striated muscle including mesenchyme and cartilage, connective tissue layers of the skin, cells from the heart, including the endocardium, myocardium, dorsal and ventral mesocardium and septum transversum, artial cells, blood vessel cells, and cells of the reproductive organs, including primordial germ cells, Sertoli cells, and follicular cells) may be reprogrammed. Additional, hematopoietic stem cells and mesenchymal stem cells described above may be isolated from patients and reprogrammed.

B. Reprogramming and Programming Cells

Methods for reprogramming cells are described in Hakelien et al., Reprogramming fibroblasts to express T-cell functions using cell extracts, Nature Biotech. 20:460-466 (2002); see also, WO 02/057,415, the entire contents of which are incorporated herein by reference. In preferred embodiments, cells are programmed or reprogrammed using a cell extract.

In some embodiments, cell extracts are preferably obtained from differentiated cells (e.g., cartilage, neurons, glial cells, pancreatic cells, liver cells, islet cells, etc.) when the goal is to induce stem cells to adopt a particular developmental fate (i.e., program the stem cells). The present invention is not limited to the programming of any particular type of stem cell. Indeed, a variety of stem cells may be programmed, including, but not limited to, embryonic, hematopoietic, or mesenchymal stem cells, as well as stem cells obtained from different species, including humans and other primates, rats, mice, rabbits, etc. In these embodiments, an extract of a target cell type is prepared as described in more detail below. It is understood that extract from cells of one species may be utilized with target cells of another species. The stem cells are then exposed to the target cells type extract under conditions such that the stem cell is induced to adopt the developmental fate of the target cell type. Thus, stem cells may be induced to adopt a variety of developmental fates, including, but not limited to, cells of endodermal origin (e.g., cells from the intestine, liver, pancreas, duodenum, small intenstine, lungs, gall bladder, etc.), ectodermal origin (e.g., cells from the skin, cornea, lens, and cranial ganglia), and mesodermal origin (e.g., cells from the urinary system including pronephros, mesonephros and metanephros cells, skeletogenous tissue and voluntary striated muscle including mesenchyme and cartilage, connective tissue layers of the skin, cells from the heart, including the endocardium, myocardium, dorsal and ventral mesocardium and septum transversum, artial cells, blood vessel cells, and cells of the reproductive organs, including primordial germ cells, Sertoli cells, and follicular cells) as well as connective tissues (i.e. the tissues of the body that support the specialized elements; particularly adipose, areolar, osseous, cartilaginous, elastic, marrow stroma, muscle, and fibrous connective tissues), lymphoid, myeloid and erythroid cells (e.g., B-cells and T-cells, monocytes, granulocytes, and red blood cells) and neurons, glia, melanocytes, cartilage and connective tissue of the head and neck, stroma of various secretory glands and cells in the outflow tract of the heart.

In other embodiments, cell extracts are preferably obtained from stem cells (e.g., embryonic, hematopoietic, or mesenchymal stem cells) when the goal is to treat target patient cells to de-differentiate them. Accordingly, it is contemplated that stem cell extracts are useful for treating cells differentiated cells obtained from a patient so that the cells are reprogrammed to an earlier developmental fate (i.e., de-differentiated). The present invention is not limited to the use of any particular stem cells extract. Indeed, a variety of stem cells extracts may be utilized, including those obtained embryonic, hematopoietic, or mesenchymal stem cells, as well as stem cells obtained from different species, including humans and other primates, rats, mice, rabbits, etc. It is understood that extract from stem cells of one species may be utilized with target cells of another species. As exemplary embodiments of de-differentiation, extracts of stem cells may be used to reprogram skin cells more closely resembling primitive epidermis. Likewise, extracts of stem cells may be used to reprogram cartilage cells to mesenchyme and mesenchymal cells to cells resembling schlerotome or even somites. As another example, stem cell extracts may be used to reprogram cells derived from the intestine, liver, or pancreas to cells resembling primitive endoderm.

In some embodiments, a second reprogramming step follows the first programming or reprogramming treatment. For example, when a stem cell extract is used to de-differentiate target a target cell population, the de-differentiated target cell population may be subsequently treated with an extract derived from a differentiated cell line so that the de-differentiated target cell population adopts the developmental fate of the differentiated cell line. It is contemplated that such methods are useful for ex vivo treatment of cells derived from a patient, which are then reintroduced into the patient. Such therapies are particularly useful because by using the patient's own cells, there should be no rejection by the immune system. In particularly preferred embodiments where it desired to produce cells of a particular developmental lineage (e.g., ectoderm, endoderm, or mesoderm), cells of a similar lineage (e.g., ectodermal, endodermal, or mesodermal origin, respectively) are de-differentiated and then treated with the extract of differentiated cells. It is contemplated that cells that are originally derived from a particular lineage serve as the best candidates for forming cells of a related lineage. For example, cells derived from the pancreas can be treated sequentially with a stem cell extract and then a liver cell extract to produce cells having characteristics of liver cells. As another example, cartilage cells can be treated sequentially with a stem cell extract and then a kidney cell extract (e.g., mesonephros cell extract) to produce cells having characteristics of kidney cells.

In other embodiments, stem cells may be sequentially treated with extracts of more than one cell type in order to induce the stem cells to adopt a particular development fate. For example, a stem cell population may first be treated with an extract of mesenchymal stem cells and then with an extract of cartilage cells in order to produce cells having the characteristics of cartilage cells. As another example, a stem cell population may first be treated with an extract of neural stem cells and then with an extract of glial cells in order to produce cells having the characteristics of glial cells. As will be noted, the present invention is not limited to the use or sequential use of any particular type of cell extracts. Indeed, the cell extracts may be obtained from embryonic sources of any species (e.g., neural crest cells from mice, rates, or humans), from established immortal or mortal cell culture lines (e.g., CHO cells, 293 cells, etc); or from living organisms (i.e., adult or young mice, rats or humans).

In preferred embodiments, the cell extracts are obtained by lysing the cells. In some embodiments, the cells are isolated and frozen in liquid nitrogen. The cells are then thawed, washed in a lysis buffer (See, e.g., Collas et al., J. Cell. Biol. 147:1167-80 (1999)), sedimented, and resuspended in 2 volumes of lysis buffer. Cells and nuclei are then disrupted with a tip sonicator (2 mm diameter) and the lysate is cleared at 15,000 g for 15 minutes at 4° C. The extracts may be either used fresh or frozen for later use. When large lots of extracts are prepared and frozen, individual aliquots can be evaluated for reprogramming or programming activity in order to promote consistency.

In preferred embodiments, cells to be reprogrammed are first permeabilized. The present invention is not limited to any particular method of permeabilization. Indeed, a variety of methods for permeabilization are contemplated, including, but not limited to, treatment with streptolysin and electroporation. In preferred embodiments, the cells are grown on polylysine coverslips in a medium appropriate for culture of the cells. The cells are preferably cultured until a density of about 50,000 to 100,000 cells per coverslip is obtained. In preferred embodiments, the cells are then treated with about 200 ng/ml streptolysin O in Ca²⁺ free Hank's balanced salt solution (Gibco-BRL) for 50 minutes at 37° C.

Fresh or frozen cell extract is then used to overlay the coverslips. In preferred embodiments, the extracts contain an ATP-generating system and 1 mM each of ATP, CTP, GTP, and UTP. In further preferred embodiments, the cells are exposed to the extract for one hour at 37° C. in air. In still further preferred embodiments, the cell membranes are then resealed by incubating the cells for about two hours at 37° C. in an appropriate medium (e.g., RPMI 1640) supplemented with 2 mM CaCl₂.

C. Use of Telomerase

The present invention also contemplates the use of telomerase or nucleic acid encoding telomerase to treat cells, either alone in conjunction with the extracts described above. Suitable telomerases are known in the art, including those described in U.S. Pat. Nos. 6,548,298; 6,545,133; 6,387,619; and 6,309,867, each of which is incorporated herein by reference. In particular, the telomerase and/or nucleic acid encoding telomerase (e.g., cDNA, mRNA or genomic DNA) can be provided in a solution in which the target cells can be permeabilized as described above. It is contemplated that treatment with telomerase will result in a lengthening of telomeres. Such lengthening may be assayed by methods known in the art, including immunocytochemistry with antibodies directed against telomers and analysis of chromosome spreads (karyotyping).

D. Engineering Cells

It is contemplated that a variety of exogenous genes may be introduced into the cell described above. In some embodiments, the exogenous genes are marker genes. Such genes are useful for identifying the cells generated by the methods described above after they are transferred back to a patient. A wide variety of useful marker genes are known in the art, Examples of reporter genes include, but are not limited to, luciferase (See, e.g., deWet et al., Mol. Cell. Biol. 7:725 [1987] and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

These marker genes may be introduced into the cells by a variety of methods, including, calcium phosphate co-precipitation, liposome mediated transfection (e.g., geneFECTOR (VennNova), retroviral vector mediated transfection, microinjection, and microparticle bombardment. In particularly preferred embodiments, GFP is inserted as a marker gene as described in U.S. Pat. No. 5,989,837, incorporated herein by reference.

In other embodiments, it is contemplated that the product of the exogenous gene has a desired biological activity. For example, for cells intended to transplanted in to the liver or pancreas, the exogenous gene is preferably one that directs the production and secretion of insulin in response to glucose levels (See, e.g., WO 00/04171, incorporated herein by reference). As another example, for cells that will be transplanted into the brain for the treatment of Parkinson's disease, the exogenous gene is preferably a gene in the dopamine production pathway. Other exogenous genes which may be introduced into the cells include, but are not limited to, epidermal growth factor, basic fibroblast growth factor, glial derived neurotrophic factor, insulin-like growth factors I and II, nuertotrphin-3, neurotrophin-4/5, ciliary neurotrophic factor, AFT-1, cytokine genes (interleukins, interferons, colony stimulating factors (alpha and beta)), and genes encoding therapeutic enzymes, collagen, human serum albumin, etc.

In other preferred embodiments, the exogenous gene encodes a selectable marker. In particular preferred embodiments, the selectable is a negative selectable marker such as thymidine kinase.

E. Cell Transplant Therapy

It is contemplated that the cell described above find use in a variety of cell transplant therapies. In particular, the cell lines described above can be differentiated into any desired cell type. In some embodiments, hematopoietic cell lines are generated from the cell lines described above and used to treat diseases that require bone marrow transplantation such as ovarian cancer and leukemia, as well as diseases that attack the immune system such as AIDS. In still other embodiments, the cell lines described above are used to generate neural cell lines. Diseases treatable by transplantation of such cell lines include Parkinson's disease, Alzheimer's disease, ALS, and cerebral palsy. Other diseases treatable by cell transplant therapy include spinal cord injuries, multiple sclerosis, muscular dystrophy, diabetes, liver diseases, heart diseases, cartilage replacement, burns, foot ulcers, and kidney diseases.

Accordingly, the present invention provides methods for transplant therapy comprising providing cell produced by the reprogramming and programming methods described above and a subject, and transplanting the cells into the subject under conditions such that said cells are incorporated into the subject. For example, in some embodiments, the cells having a neural cell phenotype are transplanted into the nervous system of a subject (e.g., brain or spinal cord). In other embodiments, the cells displaying a mesodermal phenotype are transplanted into the liver of the subject.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, developmental biology, genetics, or related fields are intended to be within the scope of the following claims.

1. A composition comprising isolated stem cells and an extract of a differentiated cell line.

2. The composition of claim 1, wherein said isolated stem cells are human stem cells.

3. The composition of claim 1, wherein said isolated stem cells are non-human stem cells.

4. The composition of claim 1, wherein said extract of a differentiated cell line is derived from a neural cell line.

5. The composition of claim 1, wherein said extract of a differentiated cell line is derived from a hematopoietic cell line.

6. The composition of claim 1, wherein said extract of a differentiated cell line is derived from a mesenchymal cell line.

7. The composition of claim 1, wherein said extract of a differentiated cell line is derived from an endodermal cell line.

8. The composition of claim 1, wherein said extract of a differentiated cell line is derived from an ectodermal cell line.

9. The composition of claim 1, wherein said extract of a differentiated cell line is derived from a mesodermal cell line.

10. A method comprising:
a) providing isolated stem cells and an extract of a differentiated cell line;
b) treating said isolated stem cells with said extract of a differentiated cell line under conditions such that said isolated stem cells adopt a differential fate approximating the differential fate of said differentiated cell line.

11. The method of claim 10, wherein said isolated stem cells are human stem cells.

12. The method of claim 10, wherein said isolated stem cells are non-human stem cells.

13. The method of claim 10, wherein said extract of a differentiated cell line is derived from a neural cell line.

14. The method of claim 10, wherein said extract of a differentiated cell line is derived from a hematopoietic cell line.

15. The method of claim 10, wherein said extract of a differentiated cell line is derived from a mesenchymal cell line.

16. The method of claim 10, wherein said extract of a differentiated cell line is derived from an endodermal cell line.

17. The method of claim 10, wherein said extract of a differentiated cell line is derived from an ectodermal cell line.

18. The method of claim 10, wherein said extract of a differentiated cell line is derived from a mesodermal cell line.

19. The method of claim 10, wherein said treating further comprising permeabilizing said isolated cells.

20. Differentiated stem cells produced by the method of claim 10.

21. Organs comprising the differentiated stem cells of claim 20.

22. The method of claim 21, wherein said isolated stem cells are genetically modified.

23. A method comprising:
a) providing isolated cells, a stem cell extract, and a differentiated cell line extract;
b) treating said isolated cells with said stem extract under conditions such that said isolated cells are reprogrammed to an earlier differential fate; and
c) treating said reprogrammed cells with said differentiated cell line extract under conditions such that said reprogrammed cells adopt a differential fate approximating the differential fate of said differentiated cell line.

24. A method comprising:
a) providing isolated cells and a solution comprising telomerase or a nucleic acid encoding telomerase;
b) permeabilizing said isolated cells in said solution; and
c) culturing said isolated cells, wherein the telomers of said cells are lengthened.

25. The method of claim 24, wherein said isolated cells are human cells.

26. The method of claim 24, wherein said telomerase is human telomerase.

27. The method of claim 24, wherein said cells are genetically modified.

28. Cells produced by the method of claim 24.

29. Organs comprising the cells of claim 28.
